SEQUENCE LISTING (25 sequences)

SEQ ID NO: 1 - protein, 245 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 116 aa, Artificial Sequence
  Synthetic
EVKLLQSGGGLVQPGGSLKLSCAASGIDFSRYWMSWVRRAPGKGLEWIGEINLDSSTKNYAPSLKDKFIISRDNAKNTLYLQMSKVRSEDTALYYCARNYVGMDYWGQGTSVTVSS

SEQ ID NO: 3 - protein, 111 aa, Artificial Sequence
  Synthetic
DIVLTQSPASLAVSLGQRATISCRASKSVSSSDYTYMHWYQQKPGQPPKLLIYLASNLESGVPARFSGSGSGTDFTLNIHPVEEEDAATYYCQHSRELPPTFGGGTKLEIK

SEQ ID NO: 4 - protein, 270 aa, Artificial Sequence
  Synthetic
MGWSCIILFLVATATGVHSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSHHHHHH

SEQ ID NO: 5 - DNA, 813 bp, Artificial Sequence
  Synthetic
atgggctggtcctgcatcattctgtttctggtggccacagccaccggcgtgcacagcgatattcagatgacccagaccaccagcagcctgtctgcctctctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagccatcaccaccaccatcactga

SEQ ID NO: 6 - protein, 19 aa, Artificial Sequence
  Synthetic
TGSTSGSGKPGSGEGSTKG

SEQ ID NO: 7 - protein, 506 aa, Artificial Sequence
  Synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 508 aa, Artificial Sequence
  Synthetic
MALPVTALLLPLALLLHAARPQVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 508 aa, Artificial Sequence
  Synthetic
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 5 aa, Artificial Sequence
  Synthetic
RYWMS

SEQ ID NO: 11 - protein, 17 aa, Artificial Sequence
  Synthetic
EINLDSSTKNYAPSLKD

SEQ ID NO: 12 - protein, 7 aa, Artificial Sequence
  Synthetic
NYVGMDY

SEQ ID NO: 13 - protein, 8 aa, Artificial Sequence
  Synthetic
SGIDFSRY

SEQ ID NO: 14 - protein, 6 aa, Artificial Sequence
  Synthetic
NLDSST

SEQ ID NO: 15 - protein, 15 aa, Artificial Sequence
  Synthetic
RASKSVSSSDYTYMH

SEQ ID NO: 16 - protein, 7 aa, Artificial Sequence
  Synthetic
LASNLES

SEQ ID NO: 17 - protein, 9 aa, Artificial Sequence
  Synthetic
QHSRELPPT

SEQ ID NO: 18 - protein, 18 aa, Artificial Sequence
  Synthetic
MDFGLIFFIVALLKGVQC

SEQ ID NO: 19 - protein, 20 aa, Artificial Sequence
  Synthetic
METDTLLLWVLLLWVPGSTG

SEQ ID NO: 20 - protein, 134 aa, Artificial Sequence
  Synthetic
MDFGLIFFIVALLKGVQCEVKLLQSGGGLVQPGGSLKLSCAASGIDFSRYWMSWVRRAPGKGLEWIGEINLDSSTKNYAPSLKDKFIISRDNAKNTLYLQMSKVRSEDTALYYCARNYVGMDYWGQGTSVTVSS

SEQ ID NO: 21 - protein, 131 aa, Artificial Sequence
  Synthetic
METDTLLLWVLLLWVPGSTGDIVLTQSPASLAVSLGQRATISCRASKSVSSSDYTYMHWYQQKPGQPPKLLIYLASNLESGVPARFSGSGSGTDFTLNIHPVEEEDAATYYCQHSRELPPTFGGGTKLEIK

SEQ ID NO: 22 - protein, 458 aa, Artificial Sequence
  Synthetic
MDFGLIFFIVALLKGVQCEVKLLQSGGGLVQPGGSLKLSCAASGIDFSRYWMSWVRRAPGKGLEWIGEINLDSSTKNYAPSLKDKFIISRDNAKNTLYLQMSKVRSEDTALYYCARNYVGMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 23 - protein, 238 aa, Artificial Sequence
  Synthetic
METDTLLLWVLLLWVPGSTGDIVLTQSPASLAVSLGQRATISCRASKSVSSSDYTYMHWYQQKPGQPPKLLIYLASNLESGVPARFSGSGSGTDFTLNIHPVEEEDAATYYCQHSRELPPTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 24 - protein, 116 aa, Artificial Sequence
  Synthetic
EVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSS

SEQ ID NO: 25 - protein, 111 aa, Artificial Sequence
  Synthetic
DIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVPYTFGGGTKLEIK

(19) United States
(12) Patent Application Publication
(10) Pub. No.: US 2023/0103885 A1
(43) Pub. Date: Apr. 6, 2023

(54) ANTI-IDIOTYPE ANTIBODIES TARGETING ANTI-CD19 CHIMERIC ANTIGEN RECEPTOR

(71) Applicant: CRISPR THERAPEUTICS AG, Zug (CH)

(72) Inventor: Lalit KUMAR, Cambridge, MA (US)

(21) Appl. No.: 17/904,002

(22) PCT Filed: Feb. 10, 2021
(86) PCT No.: PCT/IB2021/051100

§ 371 Date: Aug. 10, 2022

Related U.S. Application Data
(60) Provisional application No. 62/972,750, filed on Feb. 11, 2020.

(51) Int. Cl. C07K 16/28; G01N 33/68

(52) U.S. Cl. CPC C07K 16/2809; G01N 33/6872

(57) ABSTRACT

High affinity antibodies capable of binding to a single-chain variable fragment (scFv) of anti-CD19 antibody FMC63, for example, the scFv expressed on cell surface as a portion of a chimeric antigen receptor (CAR). Also provided herein are methods for producing such anti-scFv antibodies and methods of using the antibodies disclosed herein for detecting, for example, T cells expressing an anti-CD19 CAR that comprise the scFv as an extracellular domain.

### CROSS REFERENCE TO RELATED APPLICATIONS

This application claims the benefit of the filing dates of U.S. Provisional Application No. 62/972,750, filed Feb. 11, 2020, the entire contents of which is incorporated by reference herein.

### BACKGROUND

Chimeric antigen receptor (CAR) T-cell therapy has shown promising therapeutic effects in cancer treatment. Typically, CAR-T cells are generated by genetic engineering of either patient immune cells (autologous) or immune cells from human donors (allogenic).

Production of high-quality, clinical grade CAR-T cells is a prerequisite for the wide application of this technology. It is therefore of great interest to develop tools for detecting CAR-expressing T cells.

### SUMMARY

The present disclosure is based, at least in part, on the development of antibody 29E4B5 having high binding affinity and specificity to a single-chain variable fragment (scFv) of mouse anti-human CD19 antibody FMC63 (SEQ ID NO:1), particularly to the scFv expressed on a cell surface. For example, antibody 29E4B5 (a.k.a., 29E4B5-1) disclosed herein displayed high binding affinity and specificity to T cells expressing an anti-CD19 chimeric receptor (anti-CD19 CAR) having the scFv of SEQ ID NO:1 as the extracellular domain. Antibody 29E4B5 also displayed superior binding affinity to anti-CD19 CAR T cells compared to a reference antibody (rec_mab3) capable of binding to the same anti-CD19 CAR T cells.

Accordingly, the present disclosure provides, in some aspects, an isolated antibody, which binds a single-chain variable fragment (scFv) consisting of the amino acid sequence of SEQ ID NO: 1 (anti-scFv antibody). In some instances, the anti-scFv antibody binds the same epitope of the scFv as antibody 29E4B5 or competes against antibody 29E4B5 for binding to the scFv. In some embodiments, the isolated antibody binds the scFv expressed on a cell surface, for example, as the extracellular domain of a chimeric antigen receptor.

In some embodiments, the isolated antibody comprises the same heavy chain complementary determining regions and the same light chain complementary determining regions as exemplary antibody 29E4B5. For example, the isolated antibody may comprise the same V_{H} and the same V_{L} as antibody 29E4B5.

Any of the anti-scFv antibodies disclosed herein may be full-length antibodies. Alternatively, the anti-scFv antibodies may be an antigen-binding fragment.

In addition, the present disclosure features a nucleic acid or a set of nucleic acids (two individual nucleic acid molecules), which collectively encodes any of the anti-scFv antibodies described herein. In some embodiments, the nucleic acid or the set of nucleic acids is a vector or a set of vectors, for example, an expression vector(s).

Also provided herein is a host cell comprising the nucleic acid or the set of nucleic acids coding for any of the anti-scFv antibodies disclosed herein. In some embodiments, the host cell is a mammalian cell.

In other aspects, the present disclosure features a method for detecting or quantifying a single-chain variable fragment (scFv) that consists of the amino acid sequence of SEQ ID NO: 1. Such a method may comprise: (i) contacting an anti-scFv antibody as disclosed herein (e.g., an antibody having the same heavy chain and light chain CDRs or the same V_{H} and V_{L} chains as exemplary antibody 29E4B5 with a sample suspected of containing the scFv of SEQ ID NO:1, and (ii) detecting binding of the antibody to the scFv. In some embodiments, the scFv is the extracellular domain of an anti-CD19 chimeric antigen receptor (CAR) expressed on a cell surface. In some embodiments, the anti-scFv antibody can be conjugated to a detectable label.

In some embodiments, the sample may comprise a plurality of T cells, which are genetically engineered to express an anti-CD19 CAR that comprises the scFv of SEQ ID NO:1 as the extracellular domain. In some embodiments, the plurality of T cells may further comprise a disrupted TRAC gene, a disrupted β2M gene, or both. In some examples, the plurality of T cells are prepared from T cells obtained from one or more donors.

In some instances, the sample is derived from a manufacturing process for producing the plurality of T cells that are genetically engineered for expressing the anti-CD19 CAR.

In some examples, the sample is a biological sample obtained from a subject administered a plurality of T cells, which are genetically engineered to express the anti-CD19 CAR. In some embodiments, the sample is a blood sample. The subject may be a human cancer patient, for example, a human cancer patient having a relapsed or refractory B-cell malignancy. Exemplary B-cell malignancy includes, but is not limited to, non-Hodgkin lymphoma or B-cell lymphoma.

Further, the present disclosure provides a method of producing any of the anti-scFv antibodies disclosed herein. The method may comprise: (i) culturing any of the host cells described herein that comprise one or more nucleic acids encoding the anti-scFv antibody under conditions allowing for expression of the antibody that binds the scFv; and (ii) harvesting the antibody thus produced from the cell culture. In some embodiments, the method may further comprise (iii) purifying the antibody after step (ii).

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1B are photos showing recombinant FMC63-ScFv protein analyzed by SDS-PAGE (FIG. 1A) and Western-blot analysis (FIG. 1B). Lane M₁: Protein Marker (Takara Bio USA, Mountain View, Calif., Cat. No. 3452). Lane M₂: Protein Marker (GenScript Biotech, Piscataway, N.J., Cat. No. M00521). Lane 1: Reducing conditions. Lane 2: Non-reducing conditions. Lane P: Human IgG1, Kappa (Sigma-Aldrich, St. Louis, Mo., Cat. No. 15154) as a positive control. Primary antibody: Mouse-anti-His mAb (GenScript Biotech, Piscataway, N.J., Cat. No. A00186).

FIG. 2 is a diagram showing that antibody clone 29E4B5 binds specifically to anti-CD19 CAR T cells (CAR T cells that express a CAR containing anti-FMC63-scFv, but not anti-BCMA CAR T cells or anti-CD70 CAR T cells.

### DETAILED DESCRIPTION

Provided herein are antibodies capable of binding to a single-chain variable fragment (scFv) having the amino acid sequence of SEQ ID NO:1 (derived from mouse anti-human CD19 antibody FMC63), e.g., capable of binding to the scFv expressed on cell surface as the extracellular domain of an anti-CD19 chimeric antigen receptor (CAR). As such, the antibodies disclosed herein may be used for detecting presence of cells (e.g., T cells) expressing such an anti-CD19 CAR in a sample, e.g., samples obtained from a manufacturing process for producing anti-CD19 CAR-T cells or samples obtained from patients who are administered anti-CD19 CAR-T cells.

I. Antibodies Binding to Anti-CD19 Single-Chain Variable Fragment (scFv)

The present disclosure provides antibodies (e.g., antibody 29E4B5) binding to a single-chain variable fragment (scFv) having the amino acid sequence of SEQ ID NO: 1 (provided below), which comprises the heavy chain variable domain (V_{H}) and light chain variable domain (V_{L}) derived from mouse anti-human CD19 antibody FMC63. As such, the antibodies provided herein may be referred to as anti-scFv antibodies or anti-idiotypic (anti-ID) antibodies. In some embodiments, the antibodies disclosed herein are capable of binding to the scFv expressed on a cell surface. In specific examples, the antibodies disclosed herein bind to a cell-surface expressed anti-CD19 chimeric antigen receptor (CAR) comprising the scFv of SEQ ID NO:1 as the extracellular domain. The linker fragment is in boldface.

Amino Acid Sequence of the scFv Antigen (SEQ ID
NO: 1):
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGSTSGSGKPGSGEGSTKGEVKLQESGPGLVAPSQSLSVTCTVS
GVSLPDYGVSWIRQPPRKGLEWLGVIWGSETTYYNSALKSRLTIIKDNSK
SQVFLKMNSLQTDDTAIYYCAKHYYYGGSYAMDYWGQGTSVTVSS

An antibody (interchangeably used in plural form) is an immunoglobulin molecule capable of specific binding to a target, such as the scFv of SEQ ID NO:1 in the present application, through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of an immunoglobulin molecule that comprises an antigen recognition site of the required specificity, including glycosylation variants of antibodies, amino acid sequence variants of antibodies, and covalently modified antibodies. An antibody as disclosed herein includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.

The anti-scFv antibodies described herein may be a full-length antibody, which contains two heavy chains and two light chains, each including a variable domain and a constant domain. Alternatively, the anti-scFv antibodies described herein can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Nati. Acad. Sci. USA 85:5879-5883.

The anti-scFv antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the anti-scFv antibodies described herein, e.g., antibody 29E4B5, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-scFv antibodies described herein are human antibodies, which may be isolated from a human antibody library or generated in transgenic mice. For example, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In other embodiments, the anti-scFv antibodies described herein may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-scFv antibodies described herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, the anti-scFv antibodies described herein specifically bind to the corresponding target antigen (i.e., the anti-CD19 scFv of SEQ ID NO: 1 or a polypeptide such as a chimeric antigen receptor comprising such) or an epitope thereof. An antibody that “specifically binds” to an antigen or an epitope is a term well understood in the art. A molecule is said to exhibit “specific binding” if it reacts more frequently, more rapidly, with greater duration, with greater avidity, and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In some embodiments, the anti-scFv antibodies described herein (e.g., antibody 29E4B5) have a suitable binding affinity for the target antigen (i.e., the anti-CD19 scFv of SEQ ID NO: 1 or a polypeptide such as a chimeric antigen receptor comprising such) or antigenic epitopes thereof. As used herein, “binding affinity” refers to the apparent association constant or K_{A}. The K_{A} is the reciprocal of the dissociation constant (K_{D}). The antibody described herein may have a binding affinity (K_{D}) of at least 100 mM, 10 mM, 1 mM, 0.1 mM, 100 μM, 10 μM, 1 μM, 0.1 μM, 100 nM, 10 nM, 1 nM, 0.1 nM, or lower for the scFv from antibody FMC63. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 105 fold. In some embodiments, any of the antibodies disclosed herein may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, since sometimes it is sufficient to obtain a quantitative measurement of affinity (e.g., determined using a method such as ELISA or FACS analysis), which is proportional to K_{A}. The quantitative measurement thus can be used for comparisons, such as determining whether a higher affinity is, e.g., 2-fold higher, so as to obtain a qualitative measurement of affinity, or to obtain an inference of affinity, e.g., by activity in a functional assay, e.g., an in vitro or in vivo assay.

The structural information (heavy chain and light chain variable domains) of an exemplary antibody 29E4B5 is provided below. The heavy chain CDRs and light chain CDRs (determined by the Kabat approach; see, e.g., Kabat, E. A., et al. (1991) Sequences of Proteins of Immunological Interest, Fifth Edition, U.S. Department of Health and Human Services, NIH Publication No. 91-3242, imgt.org/IMGTindex/V-QUEST.php, and ncbi.nlm.nih.gov/igblast/) are identified in boldface. See also Table 7 below.

TABLE 1
V_{H} and V_{L} Sequences of anti-scFv antibody 29E4B5.

| | SEQ | |
|---|---|---|
| Descrip- | ID | |
| tion | NO: | Sequences (CDRs in boldface) |
| Heavy | 2 | EVKLLQSGGGLVQPGGSLKLSCAASGIDFSRYWMSWVR |
| chain | | RAPGKGLEWIGEINLDSSTKNYAPSLKDKFIISRDNAK |
| variable | | NTLYLQMSKVRSEDTALYYCARNYVGMDYWGQGTSVTV |
| (V_{H}) | | SS |
| Light | 3 | DIVLTQSPASLAVSLGQRATISCRASKSVSSSDYTYMH |
| chain | | WYQQKPGQPPKLLIYLASNLESGVPARFSGSGSGTDFT |
| variable | | LNIHPVEEEDAATYYCQHSRELPPTFGGGTKLEIK |
| (V_{L}) | | |

In some embodiments, the anti-scFv antibodies described herein bind to the same epitope in SEQ ID NO: 1 as the exemplary antibody 29E4B5 or compete against the exemplary antibody for binding to the scFv antigen (SEQ ID NO:1). An “epitope” as used herein refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residues, less than 2 non-overlapping amino acid residues, or only 1 non-overlapping amino acid residue) as the exemplary antibody. Whether two antibodies compete against each other for binding to the cognate antigen can be determined by a competition assay, which is well known in the art.

In some examples, the anti-scFv antibodies disclosed herein comprises the same V_{H} and/or V_{L} CDRs as the exemplary antibody 29E4B5. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such antibodies may have the same V_{H}, the same V_{L}, or both as compared to an exemplary antibody described herein. The heavy chain and light chain CDRs of exemplary antibody 29E4B5, determined by the various approaches as noted, are provided in Table 7 below.

Also within the scope of the present disclosure are functional variants of exemplary antibody 29E4B5. Such functional variants are substantially similar to the exemplary antibody, both structurally and functionally. A functional variant comprises substantially similar V_{H} and V_{L} CDRs as the exemplary antibody. For example, it may comprise only up to 8 (e.g., 8, 7, 6, 5, 4, 3, 2, or 1) amino acid residue variations in the total CDR regions of the antibody and binds the same epitope in SEQ ID NO: 1 with substantially similar affinity (e.g., having a K_{D} value in the same order). In some instances, the functional variants may have the same heavy chain CDR3 as the exemplary antibody, and optionally the same light chain CDR3 as the exemplary antibody. Alternatively or in addition, the functional variants may have the same heavy chain CDR2 as the exemplary antibody. Such an antibody may comprise a V_{H} fragment having CDR amino acid residue variations in only the heavy chain CDR1 as compared with the V_{H} of the exemplary antibody. In some examples, the antibody may further comprise a V_{L} fragment having the same V_{L} CDR3, and optionally the same V_{L} CDR1 or VL CDR₂ as the exemplary antibody.

In some instances, the amino acid residue variations (e.g., in one or more of the heavy chain and light chain CDRs of antibody 29E4B5) can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D.

In some embodiments, the anti-scFv antibodies disclosed herein may comprise heavy chain CDRs that are at least 80% (e.g., 85%, 90%, 95%, or 98%) identical, individually or collectively, as compared with the V_{H} CDRs of the exemplary antibody 29E4B5. Alternatively or in addition, the anti-scFv antibodies disclosed herein may comprise light chain CDRs that are at least 80% (e.g., 85%, 90%, 95%, or 98%) identical, individually or collectively, as compared with the V_{L} CDRs as the exemplary antibody 29E4B5. As used herein, “individually” means that one CDR of an antibody shares the indicated sequence identity relative to the corresponding CDR of the exemplary antibody. “Collectively” means that three V_{H} or V_{L} CDRs of an antibody in combination share the indicated sequence identity relative the corresponding three V_{H} or V_{L} CDRs of the exemplary antibody in combination.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-scFv antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

II. Preparation of Anti-Single-Chain Variable Fragment (scFv) Antibodies

The anti-scFv antibodies described herein (e.g., antibody 29E4B5) can be made by any method known in the art. See, for example, Harlow and Lane, (1998) Antibodies: A Laboratory Manual, Cold Spring Harbor Laboratory, New York.

In some embodiments, the anti-scFv antibody may be produced by the conventional hybridoma technology. The full-length anti-CD19 scFv antigen of SEQ ID NO: 1 or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-scFv monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as a source of antibodies encompasses all derivatives, progeny cells of the parent hybridomas that produce monoclonal antibodies capable of binding to SEQ ID NO: 1. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody (monoclonal or polyclonal) of interest (e.g., produced by a hybridoma cell line) may be sequenced and the polynucleotide sequence may then be cloned into a vector for expression or propagation. The sequence encoding the antibody of interest may be maintained in the vector in a host cell and the host cell can then be expanded and frozen for future use. In an alternative, the polynucleotide sequence may be used for genetic manipulation to, e.g., humanize the antibody or to improve the affinity (affinity maturation), or other characteristics of the antibody. For example, the constant region may be engineered to more resemble human constant regions to avoid immune response if the antibody is from a non-human source and is to be used in clinical trials and treatments in humans. Alternatively, or in addition, it may be desirable to genetically manipulate the antibody sequence to obtain greater affinity and/or specificity to the target antigen. It will be apparent to one of skill in the art that one or more polynucleotide changes can be made to the antibody and still maintain its binding specificity to the target antigen.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen, in which various fragments of the single-chain variable fragment (scFv) protein have been replaced (swapped) with sequences from a closely related, but antigenically distinct protein. By assessing binding of the antibody to the mutant scFv polypeptide, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some embodiments, the anti-scFv antibodies disclosed herein can be produced using the conventional recombinant technology as exemplified below.

Nucleic acids encoding the heavy and light chain of an antibody described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters (Brown, M. et al., Cell, 49:603-612 (1987)), those using the tetracycline repressor (tetR) (Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)). Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987)); Gossen and Bujard (1992); (M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(11):1811-1818, 1999). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an antibody described herein. The recombinant expression vector can be introduced into a suitable host cell (e.g., a dhfr− CHO cell) by a conventional method, e.g, calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of an antibody described herein (e.g., antibody 29E4B5) and the other encoding the light chain of the antibody described herein (e.g., antibody 29E4B5). Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-scFv antibody as described herein (e.g., antibody 29E4B5), vectors (e.g., expression vectors) containing such, and host cells comprising the vectors are within the scope of the present disclosure.

In other embodiments, the anti-scFv antibodies described herein can be single-chain antibody fragments (scFv). A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a single-chain variable fragment (scFv) of SEQ ID NO: 1, which can be identified from the library following routine procedures. Positive clones can be subjected to further screening to identify those that bind the scFv of SEQ ID NO: 1.

III. Applications of Anti-Single-Chain Variable Fragment (scFv) Antibodies

The present disclosure also provides methods for detecting or quantifying a single-chain variable fragment (scFv) consisting of the amino acid sequence of SEQ ID NO: 1 (specific to CD19) in a sample using any of the anti-scFv antibodies as described herein (e.g., antibody 29E4B5). To perform the method disclosed herein, any of the anti-scFv antibodies can be brought in contact with a sample suspected of containing a target antigen as disclosed herein—the anti-CD19 scFv of SEQ ID NO:1 or a polypeptide such as a CAR construct comprising such. In general, the term “contacting” or “in contact” refers to an exposure of the anti-scFv antibody disclosed herein with the sample suspected of containing the target antigen for a suitable period under suitable conditions sufficient for the formation of a complex between the anti-scFv antibody and the target antigen in the sample, if any. In some embodiments, the contacting is performed by capillary action in which a sample is moved across a surface of the support membrane. The antibody-antigen complex thus formed, if any, can be determined via a routine approach. Detection of such an antibody-antigen complex after the incubation is indicative of the presence of the target antigen in the sample. When needed, the amount of the antibody-antigen complex can be quantified, which is indicative of the level of the target antigen in the sample.

In some embodiments, a target antigen disclosed herein (i.e., the anti-CD19 scFv of SEQ ID NO:1 or a polypeptide comprising such) in a sample can be detected or quantified using any of the anti-scFv antibodies disclosed herein via an immunoassay. Examples of immunoassays include, without limitation, immunoblotting assay (e.g., Western blot), immunohistochemical analysis, flow cytometry assay, immunofluorescence assay (IF), enzyme linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for detecting the target antigen in a sample will be apparent to those of skill in the art.

In some examples, the anti-scFv antibodies as described herein (e.g., antibodies comprising the same heavy chain and light chain CDRs or comprising the same V_{H} and the same V_{L} as antibody 29E4B5) can be conjugated to a detectable label, which can be any agent capable of releasing a detectable signal directly or indirectly. The presence of such a detectable signal or intensity of the signal is indicative of presence or quantity of the target antigen in the sample. Alternatively, a secondary antibody specific to the anti-scFv antibody or specific to the target antigen may be used in the methods disclosed herein. For example, when the anti-scFv antibody used in the method is a full-length antibody, the secondary antibody may bind to the constant region of the anti-scFv antibody. In other instances, the secondary antibody may bind to an epitope of the target antigen that is different from the binding epitope of the anti-scFv antibody. Any of the secondary antibodies disclosed herein may be conjugated to a detectable label.

Any suitable detectable label known in the art can be used in the assay methods described herein. In some embodiments, a detectable label can be a label that directly releases a detectable signal. Examples include a fluorescent label or a dye. A fluorescent label comprises a fluorophore, which is a fluorescent chemical compound that can re-emit light upon light excitation. Examples of fluorescent label include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., Seta and Square dyes), squaraine rotaxane derivatives such as SeTau dyes, naphthalene derivatives (e.g., dansyl and prodan derivatives), coumarin derivatives, oxadiazole derivatives (e.g., pyridyloxazole, nitrobenzoxadiazole and benzoxadiazole), anthracene derivatives (e.g., anthraquinones, including DRAQ5, DRAQ7 and CyTRAK Orange), pyrene derivatives such as cascade blue, oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin). A dye can be a molecule comprising a chromophore, which is responsible for the color of the dye. In some examples, the detectable label can be fluorescein isothiocyanate (FITC), phycoerythrin (PE), biotin, Allophycocyanin (APC) or Alexa Fluor® 488.

In some embodiments, the detectable label may be a molecule that releases a detectable signal indirectly, for example, via conversion of a reagent to a product that directly releases the detectable signal. In some examples, such a detectable label may be an enzyme (e.g., β-galactosidase, HRP or AP) capable of producing a colored product from a colorless substrate.

Any of the anti-scFv antibodies disclosed herein can be used for detecting and/or quantifying cells (e.g., immune cells such as T cells) that are genetically engineered to express a chimeric antigen receptor comprising the anti-CD19 scFv of SEQ ID NO:1. As used herein, a chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. Generally, a CAR is a fusion polypeptide comprising an extracellular domain that recognizes a target antigen (e.g., a single-chain variable fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression.

The anti-CD19 CAR to be detected by any of the anti-scFv antibodies discloses herein comprise the anti-CD19 scFv of SEQ ID NO:1, which can be the extracellular domain when the anti-CD19 CAR is expressed on cell surface. In addition to the anti-CD19 scFv of SEQ ID NO:1, the anti-CD19 CAR disclosed herein may comprise an intracellular domain (e.g., the signaling domain of CD3ζ), and optionally one or more co-stimulatory domains (e.g., a co-stimulatory domain of CD28 or 4-1BB). In some instances, such an anti-CD19 CAR may further comprise a transmembrane domain (e.g., a transmembrane domain of CD8α). Optionally, the anti-CD19 CAR may further comprise a hinge domain, which may comprise up to 300 amino acids (e.g., 10 to 100 amino acids, or 5 to 20 amino acids). In some embodiments, the hinge domain may be a CD8 hinge domain. Other hinge domains may be used.

Examples of anti-CD19 CARs comprising the anti-CD19 scFv of SEQ ID NO:1 can be found in WO 2019/097305A2, the relevant disclosures of which are incorporated by reference herein for the purpose and subject matter referenced herein. In specific examples, the anti-CD19 CAR may comprise the amino acid sequence of SEQ ID NO: 7 (provided in Table 6 below).

In some embodiments, any of the anti-scFv antibodies disclosed herein can be used for measuring T cells expressing an anti-CD19 CAR comprising SEQ ID NO:1 as the extracellular domain during a manufacturing process for producing such anti-CD19 CAR T cells, for example, a manufacturing process for producing CTX110 cells. See, e.g., U.S. Provisional Application No. 62/934,991, filed on Nov. 13, 2019, the relevant disclosures of which are herein incorporated by reference for the purposes and subject matter referenced herein. CTX110 cells are a population of genetically engineered T cells expressing an anti-CD19 CAR comprising the amino acid sequence of SEQ ID NO:7 and having disrupted endogenous TRAC and β2M genes.

In some instances, a manufacturing process for producing genetically modified T cells expressing an anti-CD19 CAR comprising the anti-CD19 scFv of SEQ ID NO:1 (e.g., CTX110 cells) may involve enriching and activating T cells, which may be obtained from human donors, introducing genetic modifications into the T cells thus activated to produce T cells, at least a portion of which express the anti-CD19 CAR and the other desired genetic edits, depleting TCRαβ-expressing T cells from the population of genetically modified T cells thus produced, and harvesting the resultant anti-CD19 CAR-expressing T cells. See, e.g., U.S. Provisional Application No. 62/934,991, filed on Nov. 13, 2019, the relevant disclosures of which are herein incorporated by reference for the purposes and subject matter referenced herein.

To monitor such a manufacturing process for producing T cells expressing the desired anti-CD19 CAR, one or more samples may be obtained during any stage of the manufacturing process, e.g., before or after a nucleic acid encoding an anti-CD19 CAR comprising the scFv of SEQ ID NO:1 is introduced into T cell, or both, and the amount of anti-CD19 CAR-expressing T cells in the sample may be measured according to methods described herein. For example, a fluorescent dye-conjugated anti-scFv antibody as disclosed herein may be incubated with the one or more samples under suitable conditions for a suitable period allowing for binding of the anti-scFv antibody to the cell surface-expressed anti-CD19 CAR. The presence of level of the T cells expressing the anti-CD19 CAR can then be determined via a routine method, for example, by fluorescence-activated cell sorting (FACS).

For example, after incubating T cells with components for genetically modifying the T cells (including introducing into the cells a nucleic acid encoding the desired anti-CD19 CAR), a sample containing the resultant T cells may be obtained and the anti-scFv antibodies disclosed herein may be used to detect or quantify the portion of T cells in the sample that express the anti-CD19 CAR. Alternatively, or in addition to, one or more samples comprising the genetically modified T cells may be obtained after the depleting step for removing TCRαβ T cells, after any of in vitro expansion steps after the genetic manipulation, and/or after harvesting the resultant genetically engineered T cells. The amount of anti-CD19 CAR-expressing T cells in these samples may be determined using the anti-scFv antibody disclosed herein.

In some examples, a sample may be obtained from a population of T cells genetically engineered to express the anti-CD19 CAR disclosed herein after cryopreservation and before administration to a patient. The amount of anti-CD19 CAR-expressing T cells (CAR*T cells) in the sample can be measured using the anti-scFv antibody disclosed herein to make sure that a sufficient amount of the anti-CD19 CAR-expressing T cells is given to the patient.

In some embodiments, any of the anti-scFv antibodies disclosed herein can be used for clinical assessment of T cells expressing an anti-CD19 CAR comprising the anti-CD19 scFv of SEQ ID NO:1 (e.g., the CTX110 cells) after such CAR-T cells are administered to a subject in need of the treatment, for example, for evaluating the in vivo pharmacokinetic (PK) and/or pharmacodynamic (PD) behavior of the anti-CD19 CAR T cells.

For example, one or more biological samples may be obtained from a human patient administered T cells genetically engineered to express the anti-CD19 CAR (e.g., the CTX110 cells) at one or more time points after the administration. The level of the CAR⁺ T cells in the one or more biological samples can be measured by any of the anti-scFv antibodies disclosed herein via a conventional method, e.g., FACS. Such CAR⁺ T cell levels, e.g., at different time point after administration, may be used to analyze PK and/or PD features of the anti-CD19 CAR-T cells in that human patient. Such CAR⁺ T cell levels may also be used for assessing potential treatment efficacy in that human patient.

As used herein, a “biological sample” refers to a composition that comprises tissue, e.g., blood, plasma or protein, from a subject. A biological sample can be an initial unprocessed sample taken from a subject or a subsequently processed sample, e.g., partially purified or preserved forms. In some embodiments, multiple (e.g., at least 2, 3, 4, 5, or more) biological samples may be collected from a subject, over time or at particular time intervals, for example to assess the level of T cells expressing the anti-CD19 CAR in a human patient who has been administered such T cells.

The terms “patient,” “subject,” or “individual” may be used interchangeably and refer to a subject who needs the analysis as described herein. In some embodiments, the subject is a human patient, which has been administered a plurality of T cells, which are genetically engineered to express the anti-CD19 CAR. In some embodiments, the human patient is a cancer patient, for example, having relapsed or refractory B-cell malignancy such as non-Hodgkin lymphoma or B-cell lymphoma.

IV. Kits for Detecting Anti-CD19 scFv of SEO ID NO:1 and Anti-CD19 CAR Comprising Such

The present disclosure also provides kits for use in detecting or quantifying a single-chain variable fragment (scFv) consisting of the amino acid sequence of SEQ ID NO: 1 in a sample, such as a sample obtained from a manufacturing process for producing anti-CD19 CAR-T cells or a sample obtained from patients who are administered anti-CD19 CAR-T cells. Such kits can include one or more containers comprising an anti-scFv antibody, e.g., any of those described herein such as antibody 29E4B5.

In some embodiments, the kit can comprise instructions for use in accordance with any of the methods described herein. The included instructions can comprise a description of detecting or quantifying the scFv in a sample as described herein. Instructions supplied in the kits of the invention are typically written instructions on a label or package insert (e.g., a paper sheet included in the kit), but machine-readable instructions (e.g., instructions carried on a magnetic or optical storage disk, or available via an internet address provided in the kit) are also acceptable.

The kits of this invention are in suitable packaging. Suitable packaging includes, but is not limited to, vials, bottles, jars, flexible packaging (e.g., sealed Mylar or plastic bags), and the like. The kits may comprise one or more aliquots of an anti-scFv antibody described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Example 1. Antigen Expression and Purification

This Example reports expression and purification of a His-tagged single-chain variable fragment of a mouse anti-human CD19 monoclonal antibody (FMC63-scFv), which was subsequently used to generate antibodies against FMC63-scFv as described in Example 2.

The FMC63-scFv protein comprises, from N-terminal to C-terminal, an artificial signal peptide at the N-terminus, an anti-CD19 scFv fragment consisting of the amino acid sequence of SEQ ID NO:1, and a His-tag at the C-terminus. The amino acid sequence and the corresponding nucleic acid sequence of this FMC63-scFv protein are shown in SEQ ID NO: 4 and SEQ ID NO: 5, respectively. Sequences corresponding to the artificial signal peptide are underlined and the His-tag sequences are shown in bold.

(SEQ ID NO: 4)
MGWSCIILFLVATATGVHSDIQMTQTTSSLSASLGDRVTISCRASQDISK
YLNWYQQKPDGTVKLLIYHTSRLHSGVPSRFSGSGSGTDYSLTISNLEQE
DIATYFCQQGNTLPYTFGGGTKLEITGSTSGSGKPGSGEGSTKGEVKLQE
SGPGLVAPSQSLSVTCTVSGVSLPDYGVSWIRQPPRKGLEWLGVIWGSET
TYYNSALKSRLTIIKDNSKSQVFLKMNSLQTDDTAIYYCAKHYYYGGSYA
MDYWGQGTSVTVSSHHHHHH
(SEQ ID NO: 5)
ATGGGCTGGTCCTGCATCATTCTGTTTCTGGTGGCCACAGCCACCGGCGT
GCACAGCGATATTCAGATGACCCAGACCACCAGCAGCCTGTCTGCCTCTC
TGGGCGATAGAGTGACCATCAGCTGTAGAGCCAGCCAGGACATCAGCAAG
TACCTGAACTGGTATCAGCAGAAACCCGACGGCACCGTGAAGCTGCTGAT
CTACCACACCAGCAGACTGCACAGCGGCGTGCCAAGCAGATTTTCTGGCA
GCGGCTCTGGCACCGACTACAGCCTGACAATCAGCAACCTGGAACAAGAG
GATATCGCTACCTACTTCTGCCAGCAAGGCAACACCCTGCCTTACACCTT
TGGCGGAGGCACCAAGCTGGAAATCACCGGCTCTACAAGCGGCAGCGGCA
AACCTGGATCTGGCGAGGGATCTACCAAGGGCGAAGTGAAACTGCAAGAG
TCTGGCCCTGGACTGGTGGCCCCATCTCAGTCTCTGAGCGTGACCTGTAC
AGTCAGCGGAGTGTCCCTGCCTGATTACGGCGTGTCCTGGATCAGACAGC
CTCCTCGGAAAGGCCTGGAATGGCTGGGAGTGATCTGGGGCAGCGAGACA
ACCTACTACAACAGCGCCCTGAAGTCCCGGCTGACCATCATCAAGGACAA
CTCCAAGAGCCAGGTGTTCCTGAAGATGAACAGCCTGCAGACCGACGACA
CCGCCATCTACTATTGCGCCAAGCACTACTACTACGGCGGCAGCTACGCC
ATGGATTATTGGGGCCAGGGCACCAGCGTGACCGTGTCTAGCCATCACCA
CCACCATCACTGA

The DNA sequence corresponding to the FMC63-scFv (SEQ ID NO: 5) was subcloned into pcDNA3.4 vector, and the resulting FMC63-scFv DNA expression construct was transfected into Expi293F cells. One-liter of the Expi293F cells were cultured in suspension in a serum-free Expi293FTM expression medium (Thermo Fisher Scientific, Waltham, Mass., Cat. No. A1435101) to transiently express the recombinant FMC63-scFv protein. The cell culture supernatant was filtered and loaded onto a HisTrap® FF Crude column (GE Healthcare, Chicago, Ill., Cat.No. 17-5286-01). The expressed recombinant FMC63-scFv protein was purified and buffer exchanged for PBS (pH 7.2).

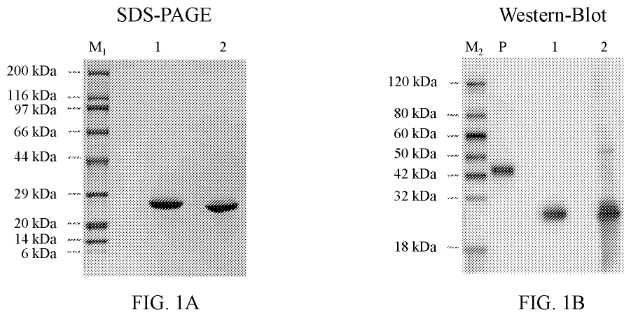

Recombinant FMC63-scFv protein was analyzed by SDS-PAGE and Western-blot under reducing (labeled 1 in FIGS. 1A-1B) and non-reducing (labeled 2 in FIGS. 1A-1B) conditions. The estimated molecular weight (MW) and purity of the recombinant FMC63-scFv protein were approximately 27 kDa and 95%, respectively. Based on Bradford protein assay, the estimated concentration and yield of the recombinant FMC63-scFv protein were 0.41 mg/ml and 11.67 mg, respectively. Mass spectrometry analysis was used to determine the experimental average MW of the purified recombinant FMC63-scFv protein. The theoretical and experimental average MWs were 27324.4 Da and 27320.2 Da, respectively. MALDI-TOF mass spectrometry was used to authenticate the amino-acid sequence of the expressed recombinant FMC63-scFv protein.

### Example 2. Anti-FMC63-scFv Antibody Generation

Immunization of mice and serum antibody titer determination was performed as described herein. Five BALB/c and five C57BL/6 mice were used for anti-FMC63-scFv antibody generation. Mice were immunized via intraperitoneal injection with FMC63-scFv protein prepared in appropriate adjuvants per the schedule shown in Table 2.

After each boost, serum was separated from the blood samples, and antibody titers were determined by indirect ELISA. The coating antigens were:

A: Recombinant FMC63-scFv protein;

(SEQ ID NO: 6)
B: TGSTSGSGKPGSGEGSTKG (FMC63-scFv linker
peptide);

C: an irrelevant His-tagged protein; and

D: Total human IgG.

The coating antigens were prepared in Phosphate Buffered Saline (PBS), pH 7.4, at 1 μg/ml and 100 d/well. The secondary antibody was Peroxidase-AffiniPure Goat Anti-Mouse IgG, Fcγ fragment-specific (Jackson ImmunoResearch, West Grove, Pa., Cat. No. 115-035-071). After the third immunization, a serum sample from each mouse was also evaluated by flow cytometry.

After the third immunization, mouse #B274 was selected for cell fusion, but no positive clones were obtained. After the fourth immunization, mouse #B279 was selected for cell fusion using a standard hybridoma protocol. Culture supernatants were subjected to ELISA screening using the same set of four antigens mentioned above. A total of six ELISA positive wells (clones) were identified from the second fusion. Table 3.

TABLE 2
Animal Immunization Schedule and Doses.

| | | (Day) | | | | | | | | | | | |
|---|---|---|---|---|---|---|---|---|---|---|---|---|---|
| | | Primary | First | First | Second | Second | Third | Third | Fourth | Fourth | Fifth | Fifth | Sixth |
| | | Immunization | Boost | Bleed | Boost | Bleed | Boost | Bleed | Boost | Bleed | Boost | Bleed | Boost |
| | No. | (day 0) | (day 14) | (day) | (day 28) | (day) | (day 47) | (day) | (day 141) | (day) | (day 215) | (day) | (day 247) |
| BALB/C | B271 | 50 μg | 25 μg | 21 | 25 μg | 35 | 25 μg | 54 | 25 μg | 148 | *25 μg | 222 | *25 μg |
| | B272 | 50 μg | 25 μg | 21 | 25 μg | 35 | 25 μg | 54 | 25 μg | 148 | *25 μg | 222 | N/A |
| | B273 | 50 μg | 25 μg | 21 | 25 μg | 35 | 25 μg | 54 | 25 μg | 148 | *25 μg | 222 | *25 μg |
| | B274 | 50 μg | 25 μg | 21 | 25 μg | 35 | 25 μg | 54 | 25 μg | N/A | N/A | N/A | N/A |
| | | | | | | | | | (day 106) | | | | |
| | B275 | 50 μg | 25 μg | 21 | 25 μg | 35 | 25 μg | 54 | 25 μg | 148 | *25 μg | 222 | N/A |
| C57BL/6 | B276 | 50 μg | 25 μg | 21 | 25 μg | 35 | 25 μg | 54 | 25 μg | 148 | *25 μg | 222 | N/A |
| | B277 | 50 μg | 25 μg | 21 | 25 μg | 35 | 25 μg | 54 | 25 μg | 148 | 25 μg | 173 | *25 μg |
| | | | | | | | | | | | (day 166) | | (day 215) |
| | B278 | 50 μg | 25 μg | 21 | 25 μg | 35 | 25 μg | 54 | 25 μg | 148 | *25 μg | 222 | N/A |
| | B279 | 50 μg | 25 μg | 21 | 25 μg | 35 | 25 μg | 54 | 25 μg | 148 | 25 μg | N/A | N/A |
| | | | | | | | | | | | (day 166) | | |
| | B280 | 50 μg | 25 μg | 21 | 25 μg | 35 | 25 μg | 54 | 25 μg | 148 | *25 μg | 222 | N/A |

*scFv-KLH rather than scFv was used for boost.

TABLE 3
ELISA Results of Hybridoma Parental Culture
Supernatants.

| | OD₄₅₀ nm with different coating | | | |
|---|---|---|---|---|
| Hybridoma Cell | antigens | | | |
| Lines | A | B | C | D |
| 17G2 | 2.3744 | 0.1341 | 0.0633 | 0.0996 |
| 29E4 | 0.6735 | 0.0711 | 0.0525 | 0.0758 |
| 33C9 | 2.0791 | 0.0685 | 0.0548 | 0.0918 |
| 41B3 | 2.422 | 0.0689 | 0.0657 | 2.0814 |
| 42A10 | 2.0192 | 0.1093 | 0.0777 | 1.365 |
| 45G2 | 2.077 | 0.0572 | 0.0502 | 0.1195 |
| Positive Control | 2.8536 | 0.0788 | 0.9391 | 2.9009 |
| (mouse #279 | | | | |
| antiserum | | | | |
| 1: 1,000) | | | | |
| Negative Control | 0.0449 | 0.0426 | 0.0468 | 0.0558 |
| (medium) | | | | |

A: Recombinant FMC63-scFv protein
B: TGSTSGSGKPGSGEGSTKG (FMC63-scFv linker peptide)(SEQ ID NO: 6)
C: His-tagged protein
D: Total human IgG

After one round of subcloning, 28 ELISA-positive subclones, representing four of the ELISA-positive clones (17G2, 29E4, 33C9 and 45G2), were obtained. Table 4.

TABLE 4
ELISA Results for Subclones of Original Four
Clones.

| | OD₄₅₀ nm with different coating | | | |
|---|---|---|---|---|
| | antigens | | | |
| Cell line | A | B | C | D |
| 17G12C10 | 2.382 | 0.115 | 0.137 | 0.108 |
| 17G12D2 | 3.121 | 0.062 | 0.068 | 0.106 |
| 17G12D10 | 2.729 | 0.073 | 0.079 | 0.112 |
| 17G12E1 | 2.388 | 0.068 | 0.058 | 0.095 |
| 17G12E3 | 2.402 | 0.072 | 0.080 | 0.136 |
| 17G12F10 | 3.006 | 0.071 | 0.053 | 0.108 |
| 17G12G7 | 2.875 | 0.062 | 0.067 | 0.113 |
| 17G12G9 | 3.195 | 0.069 | 0.056 | 0.260 |
| 17G12G10 | 2.504 | 0.110 | 0.073 | 0.136 |
| 17G12H11 | 2.623 | 0.078 | 0.097 | 0.147 |
| 29E4A2 | 1.986 | 0.049 | 0.063 | 0.063 |
| 29E4B2 | 1.493 | 0.077 | 0.109 | 0.105 |
| 29E4B5 | 1.504 | 0.087 | 0.201 | 0.178 |
| 29E4A11 | 1.531 | 0.096 | 0.110 | 0.116 |
| 29E4A12 | 1.561 | 0.091 | 0.064 | 0.072 |
| 29E4B3 | 1.588 | 0.083 | 0.086 | 0.079 |
| 29E4C5 | 1.645 | 0.096 | 0.068 | 0.069 |
| 33C9G1 | 2.036 | 0.068 | 0.377 | 0.140 |
| 33C9G2 | 2.171 | 0.072 | 0.088 | 0.119 |
| 33C9G4 | 2.212 | 0.113 | 0.095 | 0.190 |
| 33C9C5 | 2.183 | 0.102 | 0.071 | 0.103 |
| 33C9C8 | 2.079 | 0.087 | 0.062 | 0.093 |
| 33C9D1 | 2.204 | 0.102 | 0.061 | 0.089 |
| 33C9E2 | 2.088 | 0.091 | 0.060 | 0.087 |
| 45G2D9 | 2.192 | 0.104 | 0.075 | 0.232 |
| 45G2H11 | 2.353 | 0.115 | 0.083 | 0.227 |
| 45G2B10 | 2.149 | 0.118 | 0.083 | 0.154 |
| 45G2C12 | 2.661 | 0.170 | 0.111 | 0.190 |
| Positive | 1.757 | 0.274 | 0.812 | 2.733 |
| Control | | | | |
| (mouse #279 | | | | |
| antiserum | | | | |
| 1: 1,000) | | | | |
| Negative | 0.117 | 0.046 | 0.060 | 0.051 |
| Control | | | | |
| (medium) | | | | |

A: Recombinant FMC63-scFv protein
B: TGSTSGSGKPGSGEGSTKG (FMC63-scFv linker peptide)(SEQ ID NO: 6)
C: His-tagged protein
D: Total human IgG

The culture supernatants of the 28 ELISA-positive subclones were also evaluated by flow cytometry for binding to CAR T cells expressing an anti-CD19 CAR comprising the anti-CD19 scFv of SEQ ID NO:1 (anti-CD19 CAR T cells). The amino acid sequence of this anti-CD19 CAR (SEQ ID NO: 7) is provided in Table 6, and described in WO/2019/097305, the relevant disclosures of which are herein incorporated by reference for the purposes and subject matter referenced herein.

The culture supernatants of the subclones were used as primary antibody. A control anti-FMC63-scFv antibody at three different dilutions was used as a positive control. A negative culture supernatant (CS) was used as a negative control. Fluorescently labeled goat anti-mouse IgG (Jackson ImmunoResearch, West Grove, Pa., Cat. No. 115-605-008) was used as the secondary antibody.

Among the 28 subclones tested by flow cytometry, those produced from the 29E4 clone provided superior binding to anti-CD19 CAR⁺ T cells. Results from a set of representative flow cytometry profiles of the reference anti-FMC63-scFv antibody (rec_mab3) at three different dilutions and culture supernatants of three subclones (29E4A2, 29E4B2, and 29E4B5) are shown in Table 5.

TABLE 5
% CAR+ T Cells from Flow Cytometry Analysis.

| Antibody | % CAR⁺ |
|---|---|
| Secondary antibody only | 6.42% |
| control anti-FMC63-scFv antibody (1:100) | 73.4% |
| control anti-FMC63-scFv antibody (1:200) | 68.5% |
| control anti-FMC63-scFv antibody (1:400) | 66.7% |
| 29E4A2 | 71.5% |
| 29E4B2 | 71.4% |
| 29E4B5 | 72.1% |
| Negative culture supernatant (CS) | 5.14% |

The anti-FMC63-scFv antibodies from the supernatants of subclone 29E4B5 were purified at a microscale and analyzed by flow cytometry for binding to CAR T cells expressing the anti-CD19 CAR of SEQ ID NO: 7 (anti-CD19 CAR T cells). CAR T cells expressing an anti-BCMA CAR comprising an anti-BCMA scFV (anti-BCMA CAR T cells) or an anti-CD70 CAR comprising an anti-CD70 scFV (anti-CD70 CAR T cells) were used as negative controls. Anti-FMC63-scFv antibodies were analyzed at various dilutions.

Sequences of anti-BCMA CAR (SEQ ID NO: 8) and the anti-CD70 CAR (SEQ ID NO: 9) are provided in Table 6, and described in WO/2019/097305, and WO2019215500, the relevant disclosures of which are herein incorporated by reference for the purposes and subject matter referenced herein.

TABLE 6
CAR Sequences.

| | SEQ | |
|---|---|---|
| | ID | |
| CAR | NO: | Amino Acid Sequence |
| Anti- | 7 | MLLLVTSLLLCELPHPAFLLIPDIQMTQTTSSLSASLGDRV |
| CD19 | | TISCRASQDISKYLNWYQQKPDGTVKLLIYHTSRLHSGVPS |
| CAR | | RFSGSGSGTDYSLTISNLEQEDIATYFCQQGNTLPYTFGGG |
| | | TKLEITGSTSGSGKPGSGEGSTKGEVKLQESGPGLVAPSQS |
| | | LSVTCTVSGVSLPDYGVSWIRQPPRKGLEWLGVIWGSETTY |
| | | YNSALKSRLTIIKDNSKSQVFLKMNSLQTDDTAIYYCAKHY |
| | | YYGGSYAMDYWGQGTSVTVSSAAAFVPVFLPAKPTTTPAPR |
| | | PPTPAPTIASQPLSLRPEACRPAAGGAVHTRGLDFACDIYI |
| | | WAPLAGTCGVLLLSLVITLYCNHRNRSKRSRLLHSDYMNMT |
| | | PRRPGPTRKHYQPYAPPRDFAAYRSRVKFSRSADAPAYQQG |
| | | QNQLYNELNLGRREEYDVLDKRRGRDPEMGGKPRRKNPQEG |
| | | LYNELQKDKMAEAYSEIGMKGERRRGKGHDGLYQGLSTATK |
| | | DTYDALHMQALPPR |
| Anti- | 8 | MALPVTALLLPLALLLHAARPQVQLVQSGAELKKPGASVKV |
| BCMA | | SCKASGNTLTNYVIHWVRQAPGQRLEWMGYILPYNDLTKYS |
| CAR | | QKFQGRVTITRDKSASTAYMELSSLRSEDTAVYYCTRWDWD |
| | | GFFDPWGQGTTVTVSSGGGGSGGGGSGGGGSEIVMTQSPAT |
| | | LSVSPGERASISCRASQSLVHSNGNTHLHWYQQRPGQAPRL |
| | | LIYSVSNRFSEVPARFSGSGSGTDFTLTISSVESEDFAVYY |
| | | CSQTSHIPYTFGGGTKLEIKSAAAFVPVFLPAKPTTTPAPR |
| | | PPTPAPTIASQPLSLRPEACRPAAGGAVHTRGLDFACDIYI |
| | | WAPLAGTCGVLLLSLVITLYCNHRNRKRGRKKLLYIFKQPF |
| | | MRPVQTTQEEDGCSCRFPEEEEGGCELRVKFSRSADAPAYQ |
| | | QGQNQLYNELNLGRREEYDVLDKRRGRDPEMGGKPRRKNPQ |
| | | EGLYNELQKDKMAEAYSEIGMKGERRRGKGHDGLYQGLSTA |
| | | TKDTYDALHMQALPPR |
| Anti- | 9 | MALPVTALLLPLALLLHAARPQVQLVQSGAEVKKPGASVKV |
| CD70 | | SCKASGYTFTNYGMNWVRQAPGQGLKWMGWINTYTGEPTYA |
| CAR | | DAFKGRVTMTRDTSISTAYMELSRLRSDDTAVYYCARDYGD |
| | | YGMDYWGQGTTVTVSSGGGGSGGGGSGGGGSGDIVMTQSPD |
| | | SLAVSLGERATINCRASKSVSTSGYSFMHWYQQKPGQPPKL |
| | | LIYLASNLESGVPDRFSGSGSGTDFTLTISSLQAEDVAVYY |
| | | CQHSREVPWTFGQGTKVEIKSAAAFVPVFLPAKPTTTPAPR |
| | | PPTPAPTIASQPLSLRPEACRPAAGGAVHTRGLDFACDIYI |
| | | WAPLAGTCGVLLLSLVITLYCNHRNRKRGRKKLLYIFKQPF |
| | | MRPVQTTQEEDGCSCRFPEEEEGGCELRVKFSRSADAPAYQ |
| | | QGQNQLYNELNLGRREEYDVLDKRRGRDPEMGGKPRRKNPQ |
| | | EGLYNELQKDKMAEAYSEIGMKGERRRGKGHDGLYQGLSTA |
| | | TKDTYDALHMQALPPR |

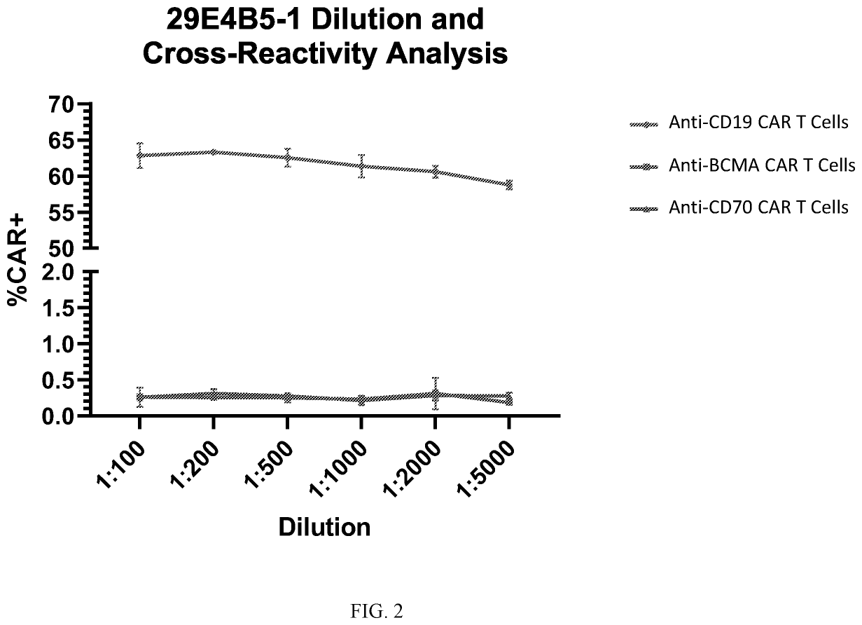

As shown in FIG. 2, the anti-FMC63-scFv antibodies from subclone 29E4B5 bound specifically to CAR T cells expressing the anti-CD19 CAR. No appreciable binding was observed between the anti-FMC63-scFv antibodies from subclone 29E4B5 to CAR T cells expressing the anti-BCMA CAR or the anti-CD70 CAR. FIG. 2.

The variable region of the mouse anti-FMC63-scFv monoclonal antibody 29E4B5 was sequenced. Total RNA was isolated from the hybridoma cells using the TRIZOL® Reagent (Thermo Fisher Scientific, Waltham, Mass., Cat. No. 15596-026). cDNA was generated by reverse-transcription using the total RNA as a template and isotype-specific anti-sense primers or universal primers. The PrimeScript™ 1^{st} Strand cDNA Synthesis Kit (Takara Bio USA, Mountain View, Calif., Cat. No. 6215A) was used according to the manufacturer's technical manual. The heavy chain and light chain sequences were amplified using rapid amplification of cDNA ends (RACE) (GenScript Biotech, Piscataway, N.J.). The amplified antibody fragments were subcloned. PCR was used to identify clones with the correct insert size. The heavy chain variable (V_{H}) domain and the light chain variable (V_{L}) domain sequences were annotated using online tools: National Center for Biotechnology Information (NCBI) Nucleotide BLAST®, IMGT/V Quest and NCBI IgBLAST®. The isotype for the 29E4B5 antibody was determined to be IgG1, κ.

The heavy chain variable (V_{H}) domain and the light chain variable (V_{L}) domain sequences of the mouse anti-FMC63-scFv monoclonal antibody 29E4B5 are provided in Table 7.

TABLE 7
Amino acid sequences of anti-scFv antibody 29E4B5.

| | | SEQ ID | Amino Acid Sequence |
|---|---|---|---|
| Kabat | HCDR1 | NO: 10 | RYWMS |
| | HCDR2 | NO: 11 | EINLDSSTKNYAPSLKD |
| | HCDR3 | NO: 12 | NYVGMDY |
| Chothia | HCDR1 | NO: 13 | SGIDFSRY |
| | HCDR2 | NO: 14 | NLDSST |
| | HCDR3 | NO: 12 | NYVGMDY |
| Kabat or | LCDR1 | NO: 15 | RASKSVSSSDYTYMH |
| Chothia | LCDR2 | NO: 16 | LASNLES |
| | LCDR3 | NO: 17 | QHSRELPPT |
| Signal | V_{H} | NO: 18 | MDFGLIFFIVALLKGVQC |
| Peptide | V_{L} | NO: 19 | METDTLLLWVLLLWVPGSTG |

| | | |
|---|---|---|
| V_{H} | NO: 2 | EVKLLQSGGGLVQPGGSLKLSCAASGIDFSRYWMSWVRRAPGK |
| | | GLEWIGEINLDSSTKNYAPSLKDKFIISRDNAKNTLYLQMSKV |
| | | RSEDTALYYCARNYVGMDYWGQGTSVTVSS |
| V_{L} | NO: 3 | DIVLTQSPASLAVSLGQRATISCRASKSVSSSDYTYMHWYQQK |
| | | PGQPPKLLIYLASNLESGVPARFSGSGSGTDFTLNIHPVEEED |
| | | AATYYCQHSRELPPTFGGGTKLEIK |
| V_{H} | NO: 20 | MDFGLIFFIVALLKGVQCEVKLLQSGGGLVQPGGSLKLSCAAS |
| Peptide, underlined) | | GIDFSRYWMSWVRRAPGKGLEWIGEINLDSSTKNYAPSLKDKF |
| (Including Signal | | IISRDNAKNTLYLQMSKVRSEDTALYYCARNYVGMDYWGQGTS |
| | | VTVSS |
| V_{L} | NO: 21 | METDTLLLWVLLLWVPGSTGDIVLTQSPASLAVSLGQRATISC |
| Peptide, underlined) | | RASKSVSSSDYTYMHWYQQKPGQPPKLLIYLASNLESGVPARF |
| (Including Signal | | SGSGSGTDFTLNIHPVEEEDAATYYCQHSRELPPTFGGGTKLE |
| | | IK |
| Heavy Chain | NO: 22 | MDFGLIFFIVALLKGVQCEVKLLQSGGGLVQPGGSLKLSCAAS |
| | | GIDFSRYWMSWVRRAPGKGLEWIGEINLDSSTKNYAPSLKDKF |
| | | IISRDNAKNTLYLQMSKVRSEDTALYYCARNYVGMDYWGQGTS |
| | | VTVSSAKTTPPSVYPLAPGSAAQTNSMVTLGCLVKGYFPEPVT |
| | | VTWNSGSLSSGVHTFPAVLQSDLYTLSSSVTVPSSTWPSQTVT |
| | | CNVAHPASSTKVDKKIVPRDCGCKPCICTVPEVSSVFIFPPKP |
| | | KDVLTITLTPKVTCVVVDISKDDPEVQFSWFVDDVEVHTAQTK |
| | | PREEQINSTFRSVSELPIMHQDWLNGKEFKCRVNSAAFPAPIE |
| | | KTISKTKGRPKAPQVYTIPPPKEQMAKDKVSLTCMITNFFPED |
| | | ITVEWQWNGQPAENYKNTQPIMDTDGSYFVYSKLNVQKSNWEA |
| | | GNTFTCSVLHEGLHNHHTEKSLSHSPGK |
| Light Chain | NO: 23 | METDTLLLWVLLLWVPGSTGDIVLTQSPASLAVSLGQRATISC |
| | | RASKSVSSSDYTYMHWYQQKPGQPPKLLIYLASNLESGVPARF |
| | | SGSGSGTDFTLNIHPVEEEDAATYYCQHSRELPPTFGGGTKLE |
| | | IKRADAAPTVSIFPPSSEQLTSGGASVVCFLNNFYPRDINVKW |
| | | KIDGSERQNGVLNSWTDQDSKDSTYSMSSTLTLTKDEYERHNS |
| | | YTCEATHKTSTSPIVKSFNRNEC |

Taken together, the results described herein demonstrate generation of antibodies against the scFv of mouse anti-human CD19 antibody (FMC63), including generation of mouse anti-FMC63-scFv monoclonal antibody 29E4B5.

### Example 3. Large Scale Antibody Production

The 29E4B5 antibody was prepared in large scales using two different methods.

In the first (native) method, hybridoma cells (29E4B5) were cultured in low IgG culture medium in a roller bottle for 10 days. The supernatants were collected and protein A purified to obtain purified antibodies. The purified antibodies were analyzed for the ability to bind the FMC63-scFv protein and anti-CD19 CAR T cells using ELISA (Table 8). The titer for monoclonal antibody 29E4B5 against the recombinant FMC63-scFv protein was estimated to be 1:512,000 (Table 8). The 29E4B5 antibody showed minimum cross activity to the FMC63-scFv linker peptide, the His tagged protein, or total human IgG.

TABLE 8
ELISA Results (OD₄₅₀) of the Purified Native Anti-FMAC63-scFV Mouse Monoclonal Antibody.

| | Concentration | 1,000 | 500 | 250 | 125 | 62.50 | 31.25 | 15.62 | 7.81 | 3.90 | 1.95 | | |
|---|---|---|---|---|---|---|---|---|---|---|---|---|---|
| Coat- | (ng/ml) | 1: | 1: | 1: | 1: | 1: | 1: | 1: | 1: | 1: | 1: | | |
| ing | Dilution | 1,000 | 2,000 | 4,000 | 8,000 | 16,000 | 32,000 | 64,000 | 128,000 | 256,000 | 512,000 | Blank | Titer |
| A | 29E4B5-1 | 1.880 | 1.891 | 1.850 | 1.789 | 1.706 | 1.317 | 0.983 | 0.676 | 0.407 | 0.245 | 0.073 | 1: 512,000 |
| B | | 0.064 | 0.059 | 0.062 | 0.059 | 0.075 | 0.061 | 0.067 | 0.076 | 0.068 | 0.077 | 0.070 | <1: 1,000 |
| C | | 0.059 | 0.059 | 0.054 | 0.057 | 0.051 | 0.059 | 0.088 | 0.060 | 0.084 | 0.085 | 0.064 | <1: 1,000 |
| D | | 0.073 | 0.080 | 0.079 | 0.069 | 0.071 | 0.076 | 0.067 | 0.064 | 0.073 | 0.076 | 0.079 | <1: 1,000 |

A: Recombinant FMC63-scFv protein
B: TGSTSGSGKPGSGEGSTKG (FMC63-scFv linker peptide)(SEQ ID NO: 6)
C: His-tagged protein
D: Total human IgG
The titer was the highest dilution where the Signal/Blank (S/B) ratio was ≥2.1.
OD₄₅₀ for Blank was the average of two technical replicates.
The starting concentration was 1 mg/ml, and the corresponding dilution ratio was calculated based on the actual concentration.

In the second (recombinant) method, a plasmid with the V_{H} and V_{L} sequences of the 29E4B5 antibody was generated and transiently transfected into HEK293 6E cells. The supernatants of the transfected HEK293 6E cells were used for large-scale purification of the recombinant 29E4B5 antibody.

The 29E4B5 antibodies produced using the first or the second method were compared with a reference anti-FMC63-scFv antibody (rec_mab3) for the ability to bind to the anti-CD19 CAR T cells using flow cytometry. Both methods produced 29E4B5 antibodies with higher affinity to the anti-CD19 cells than rec_mab3, showing higher CAR positive percentage even at 1:6,400 dilutions (Table 9).

TABLE 9
% CAR⁺ T Cells from Flow Cytometry Analysis.

| Antibody | Dilution | % CAR⁺ |
|---|---|---|
| Reference (Rec_mab3) | 1:50 | 69.8% |
| | 1:100 | 69.4% |
| | 1:200 | 69.7% |
| | 1:400 | 68.9% |
| | 1:800 | 66.4% |
| | 1:1600 | 62.2% |
| | 1:3200 | 56.6% |
| | 1:6400 | 49.1% |
| 29E4B5-1 (Native) | 1:50 | 70.1% |
| | 1:100 | 69.2% |
| | 1:200 | 67.9% |
| | 1:400 | 67.6% |
| | 1:800 | 67.6% |
| | 1:1600 | 67.9% |
| | 1:3200 | 62.9% |
| | 1:6400 | 60.5% |
| 29E4B5-1 (Recombinant) | 1:50 | 69.6% |
| | 1:100 | 69.3% |
| | 1:200 | 68.8% |
| | 1:400 | 69.1% |
| | 1:800 | 68.0% |
| | 1:1600 | 65.9% |
| | 1:3200 | 62.5% |
| | 1:6400 | 58.0% |

The amino acid sequences of the V_{H} and V_{L} of the reference anti-FMC63-scFv antibody (rec_mab3) are shown in Table 10.

TABLE 10
V_{H} and V_{L} of the reference anti-FMC63-scFv anti-
body (rec_mab3).

| | SEQ | |
|---|---|---|
| | ID | |
| | NO: | Amino Acid Sequence |
| V_{H} | 24 | EVKLVESGGGLVQPGGSLKLSCAASGFDFSRYWMSWVRQAPGKG |
| | | LEWIGEINLDSSTINYTPSLKDKFIISRDNAKNTLYLQMSKVRS |
| | | EDTALYYCARRYDAMDYWGQGTSVTVSS |
| V_{L} | 25 | DIVLTQSPASLAVSLGQRATISCRASESVDDYGISFMNWFQQKP |
| | | GQPPKLLIYAAPNQGSGVPARFSGSGSGTDFSLNIHPMEEDDTA |
| | | MYFCQQSKDVPYTFGGGTKLEIK |

Taken together, these results demonstrate that mouse monoclonal antibody (29E4B5) binds with higher affinity to T cells expressing a CAR comprising a FMC63-scFv (anti-CD19 CAR T cells) than a reference antibody (rec_mab3) in a flow cytometry assay.

### Example 4 Measurement of Anti-CD19 CAR-Expressing Cells Mixed with PBMCs

Genetically engineered T cells expressing an anti-CD19 CAR were mixed with PBMCs at 0.0%, 0.1%, 1%, 10%, 25%, 50% and 100% dilution. The genetically engineered T cells exhibit approximately 50% CAR expression, thus the expected % CAR⁺ T cells when mixed with PBMCs is: 0.0%, 0.05%, 0.5%, 5%, 12.5%, 25% and 50%. The actual percentage of CAR⁺ cells in the mixed cell population was evaluated using flow cytometry in technical duplicates using an exemplary anti-CD19 CAR anti-idiotypic antibody, 29E4B5-1, at 1:200 dilution.

As shown in Table 11, the observed percentage of CAR⁺ cells measured by flow was highly correlated to the expected percentage of anti-CD19 CAR expressing T cells when mixed in PBMCs, suggesting that the anti-CD19 CAR anti-idiotypic antibody allows for the detection and the quantification of CAR⁺ cells when mixed with PBMCs. The anti-idiotype antibody effectively detects and quantifies anti-CD19 CAR expressing cells in PMBCs, even when highly diluted (e.g., 1%). These results indicate that the anti-CD19 CAR idiotypic antibodies disclosed herein can be used to measure levels of the anti-CD19 CAR expressing cells in blood samples.

TABLE 11
Detection of Anti-CD19 CAR+ T Cells in Mixtures with PBMCs

Observed % CAR+ cells

| Expected % CAR+ cells | Gated on CD4+ | Gated on CD8+ |
|---|---|---|
| 50 | 48.9 | 44.2 |
| 25 | 29.8 | 31.6 |
| 12.5 | 16.1 | 16.6 |
| 5 | 7.07 | 8.51 |
| 0.5 | 1.21 | 1.31 |
| 0.05 | 0.34 | 0.076 |
| 0 | 0.19 | 0.11 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An isolated antibody, which binds a single-chain variable fragment (scFv) consisting of the amino acid sequence of SEQ ID NO: 1, wherein the antibody binds the same epitope of the scFv as antibody 29E4B5 or competes against antibody 29E4B5 for binding to the scFv.

2. The isolated antibody of claim 1, wherein the antibody binds the scFv expressed on a cell surface.

3. The isolated antibody of claim 1, which comprises the same heavy chain complementary determining regions and the same light chain complementary determining regions as antibody 29E4B5.

4. The isolated antibody of claim 3, which comprises the same V_{H} and the same V_{L} as antibody 29E4B5.

5. The isolated antibody of claim 1, wherein the antibody is a full-length antibody or an antigen-binding fragment thereof.

6. A nucleic acid or a set of nucleic acids, which collectively encodes an antibody of claim 1.

7. The nucleic acid or the set of nucleic acids of claim 6, which is a vector or a set of vectors.

8. The nucleic acid or the set of nucleic acids or claim 7, wherein the vector(s) is an expression vector(s).

9. A host cell comprising the nucleic acid or the set of nucleic acids of claim 6.

10. The host cell of claim 9, wherein the host cell is a mammalian cell.

11. A method for detecting or quantifying a single-chain variable fragment (scFv) that consists of the amino acid sequence of SEQ ID NO: 1 in a sample, the method comprising:
(i) contacting an antibody of claim 1 with a sample suspected of containing the scFv, and
(ii) detecting binding of the antibody to the scFv.

12. The method of claim 11, wherein the antibody is conjugated to a detectable label.

13. The method of claim 11, wherein the scFv is the extracellular domain of an anti-CD19 chimeric antigen receptor (CAR) expressed on a cell surface.

14. The method of claim 13, wherein the sample comprises a plurality of T cells, which are genetically engineered to express the anti-CD19 CAR.

15. The method of claim 14, wherein the plurality of T cells are prepared from T cells obtained from one or more donors.

16. The method of claim 14, wherein the sample is obtained from a process for producing a plurality of T cells, which are genetically engineered to express the anti-CD19 CAR.

17. The method of claim 14, wherein the sample is a biological sample obtained from a subject administered a plurality of T cells, which are genetically engineered to express the anti-CD19 CAR.

18. The method of claim 17, wherein the sample is a blood sample.

19. The method of claim 17, wherein the subject is a human cancer patient.

20. The method of claim 19, wherein the human cancer patient has a relapsed or refractory B-cell malignancy.

21. The method of claim 20, wherein the relapsed or refractory B-cell malignancy is non-Hodgkin lymphoma or B-cell lymphoma.

22. The method of claim 14, wherein the plurality of T cells comprises a disrupted TRAC gene, a disrupted β2M gene, or both.

23. A method of producing an antibody binding to a single-chain variable fragment (scFv) consisting of the amino acid sequence of SEQ ID NO: 1, the method comprising:
(i) culturing the host cell of claim 9 under conditions allowing for expression of the antibody that binds the scFv; and
(ii) harvesting the antibody thus produced from the cell culture.

24. The method of claim 23, further comprising (iii) purifying the antibody after step (ii).
